(19) United States
(12) Patent Application Publication
(10) Pub. No.: US 2019/0350669 A1
(43) Pub. Date: Nov. 21, 2019

(54) ILLUMINATION DEVICE FOR EAR SURGERY

(71) Applicant: ILLUMENEAR AS, Oslo (NO)

(72) Inventor: Marit Aarvaag Storaker, Oslo (NO)

(21) Appl. No.: 16/483,456

(22) PCT Filed: Feb. 6, 2018
(86) PCT No.: PCT/EP2018/052975

§ 371 Date: Aug. 5, 2019

(30) Foreign Application Priority Data
Feb. 9, 2017 (GB) 1702151.0

(51) Int. Cl. A61B 90/30; A61B 90/35; A61F 11/00

(52) U.S. Cl. CPC A61B 90/30; A61B 90/35; A61B 2090/306; A61B 2090/309; A61F 11/004

(57) ABSTRACT

An illumination device 1, 10, 100, 1′, 1′ for ear surgery, said illumination device 1, 10, 100, 1′, 1″ comprising a body 20, 200, 2000, 20′, 20″ having a first end 20a, 200a, 2000a, 20a′, 20a″ and a second end 20b, 200b, 2000b, 20b′, 20b″; a light source 22,220,2200, 22′, 22″ provided at the second end 20b, 200b, 2000b, 20b′, 20b″; and means 30, 300, 3000, 30′, 30″ for supporting said body 20, 200, 2000, 20′, 20″ and said light source 22, 220, 2200, 22′, 22″ in a patient's ear canal.

### TECHNICAL FIELD

The present invention relates to an illumination device for ear surgery. In particular, the illumination device may be used during surgery that involves the mastoid.

### BACKGROUND

Generally, light devices for surgeries are known in the art. Over the years, developments have provided light sources that can allow a surgeon to view various portions of a patient's body during surgery. However, some operation procedures are more complex than others and, therefore, some of these illumination devices may be too bulky for certain procedures, or the heat dissipated from the light source may be too high and harm the patient.

Surgical lights and surgical light systems illuminate a local area or cavity of a patient. Some surgical light systems in the art use external microscopic light sources that allow the surgeon to illuminate in the direction—i.e., parallel—to the line of sight. However, these devices provide a point source of light only in the direction of the line of sight of the surgeon and do not provide a reliable light source for the surgeon during surgery for other areas of the cavity of the patient. Surgical lights can, in external environments, emit powerful light, but also emit a high amount of heat. They cannot, however, provide a reliable point light source in mastoid surgery, as they are restricted in movement.

Ear surgery is very delicate in the medical field. There are several different techniques that provide access to different parts of the ear. Mastoid surgery is particularly challenging as it is a delicate procedure and requires a high level of skill to reach, and operate on, this particular part of the ear. The mastoid is situated behind the ear canal. It is a normal practice to enter the middle and inner ear through the mastoid.

There are different reasons to operate through the mastoid, or to perform a mastoidectomy. For example, to remove disease in the mastoid bone tissue, to insert cochlea implants in the inner ear through the middle ear and/or to remove tumours in the middle ear, such as tumours of the facial nerve that extends through the middle ear. In normal practice, the surgeons who carry out these surgeries rely on external light sources that illuminate the operation field. However, the external light will not illuminate all of the important and complex structures that the surgeon needs to see in order to conduct the operation with a high degree of success. All surgeries of this nature are conducted in a very small environment and it is normal that the sites, or any cavities, are 1 cm in diameter, or less.

### SUMMARY OF THE INVENTION

In one aspect, there is provided an illumination device for ear surgery. The illumination device includes a body having a first end and a second end, and a light source provided at the second end. The device also includes means for supporting the body and the light source in a patient's ear canal.

In a preferred embodiment, the means for supporting the body is provided around a portion of the body between the first end and the second end.

Preferably, the body is flexible or rigid.

In a preferred embodiment, the body is provided in a bore of the means for supporting the body and the light source.

In a preferred embodiment, the body has a longitudinal axis. The body may rotate around the longitudinal axis.

Preferably, there is provided means for deflecting/reflecting light provided at the second end of the body and along a side surface of the light source. In a preferred embodiment, the means for deflecting/reflecting light covers a portion of a top surface of the light source. Preferably, the means for deflecting/reflecting light covers between 1-99% of the top surface of the light source.

In a preferred embodiment, the first end of the body is attached to a power source to activate the light source.

Preferably, the means for supporting the body and the light source is a plug. In a preferred embodiment, the plug is made of a polymeric material.

Preferably, the light source is an LED or a fibre optic.

Preferably, the means for supporting the body is movable with respect to the body.

Preferably, the means for supporting the body is positioned external to the ear canal.

In another aspect of the present invention, there is provided a kit. The kit includes a plug adapted to fit within or external to a patient's ear canal, the plug having a bore through its centre. There is also provided a body having a first end and a second end, the body having a light source at its second end. The body is adapted to be inserted through the bore of the plug.

### BRIEF DESCRIPTION OF THE DRAWINGS

FIG. 1 shows an illumination device, in use, in accordance with one embodiment of the present invention.

FIGS. 1A and 1B show representations of the device of FIG. 1.

FIG. 2 shows an illumination device, in use, in accordance with another embodiment of the present invention.

FIGS. 2A and 2B show representations of the device of FIG. 2.

FIG. 3 shows an illumination device in accordance with a further embodiment of the present invention.

FIGS. 3A and 3B show representations of the device of FIG. 3.

FIGS. 4A and 4B show an illumination device in accordance with a further embodiment of the present invention.

FIGS. 5A and 5B show an illumination device in accordance with a further embodiment of the present invention.

### DETAILED DESCRIPTION OF THE INVENTION

Generally, there is described an illumination device for assisting in ear surgery. The present invention, in all embodiments, provides a surgeon with an illuminated area that allows for the surgeon to see during a surgery involving, for example, the ear, in particular, the mastoid. In essence, the illumination device acts as a backlight in the ear canal that illuminates the mastoid to allow for the surgeon to conduct the surgery with more precision and ease. The illumination device of the present invention also provides for low heat during surgery so as not to cause harm or tissue damage to the patient during, or after, surgery.

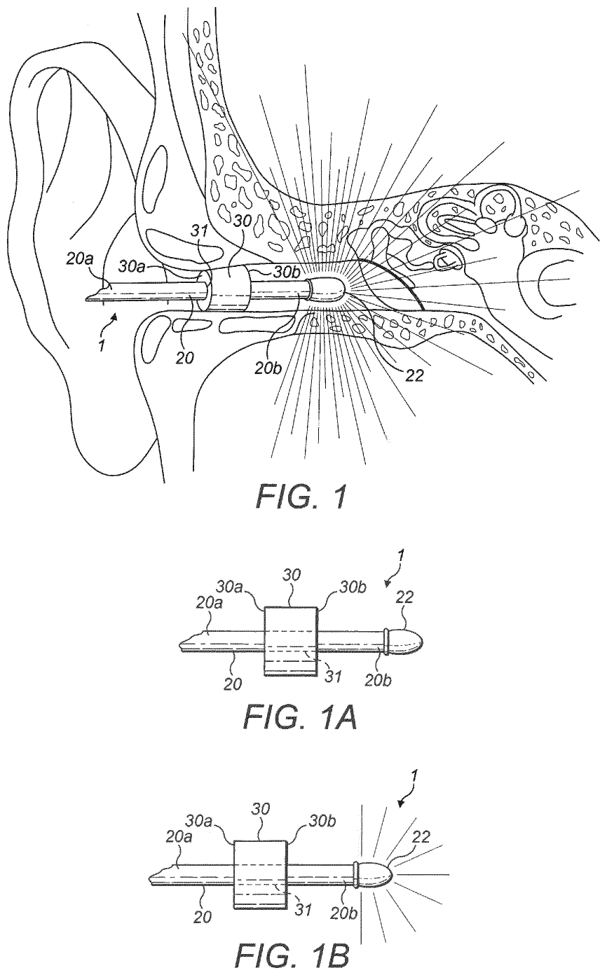

FIG. 1 shows an embodiment of such an illumination device, shown generally as reference 1 in FIG. 1. The illumination device 1 includes a body 20 having a first end 20a and a second end 20b. Arranged at, or assembled on, the second end 20b is a light source 22. The first end 20a of the body 20 can lead to a power source (not shown). The light source 22 and body 20 are of such dimensions that they can fit in an ear canal. As an example, the body 20 is a cylindrical lead and has a radius of 0.1-0.4 cm to allow the lead to enter into the ear canal. The light source 22 may also be cylindrical and also have a radius of 0.1-0.4 cm to allow the light source 22 to enter into the ear canal. Of course, the body 20 and light source 22 could be of any shape that allows for the body 20 and light source 22 to enter into the ear canal—e.g. conical, oblong etc. The body 20 may be made from polymer. The body 20 may be flexible or rigid, depending on the material from which it is made. This allows for the body 20 to be easily inserted into the area where a light source is required.

As mentioned above, there is provided a light source 22 at the second end 20b of the body 20. It is envisaged that the first end 20a is connected to a power source (not shown), and that there is provided electrical communication, through the body 20 from the first end 20a to the second end 20b to activate the light source 22. The power source at the first end may be any source that provides electrical power to the light source 22. For example, this could be a battery or a power pack. In another example, the power source may be a direct connection to a main grid. Any other sources that provide power may also be used.

The light source 22 may be, for example, an LED. In other examples, the light source 22 may be an organic LED (OLED), a light emitting electrochemical cell (LEC), a single-die LED, a high-output LED (HO-LED) etc. Alternatively, the light source 22 may be a fibre optic system that is provided through the body 20 from the power source at the first end 20a and to the second end 20b. The light source 22 may also be a combination of LED light source described above and/or the fibre optics. It is also envisaged that there may be more than one LED light source provided at the second end 20b. Of course, any other suitable light source may be provided at the second end 20b of the body 20. Additionally, it is possible to also provide any light source described above that also changes colour.

As shown in FIG. 1, there may also be provided a piece of material surrounding a portion of the body 20. This piece of material may be arranged on the body 20 to act as a means for supporting the body 20 and light source 22 when it is placed within the patient's ear canal. For the purposes of the following description, this piece of material is defined as a ‘plug’ 30. As shown in FIG. 1, the plug 30 is arranged at a portion between the first end 20a and the second end 20b of the body 20. The plug 30 is arranged to fit snug within the ear canal (i.e., to fit against the ear canal walls) so that the body 20 and light source 22 are supported on insertion into the area in which there is to be provided light.

The plug 30 may be made from rubber or other polymeric material, foam, memory foam or silicone, for example. The plug 30 may be deformable so as to fit into, and shape to, various ear canal dimensions. This allows for the plug 30 to be able to fit in ear canals for various patients and does not require a specific plug to be made for a specific patient. The plug 30 may also include a bore 31 located at its centre in order to receive the body 20. The bore 31 is sized and shaped to snugly receive the body 20. As shown in FIG. 1, the body 20 is provided through the bore 31 of the plug 30 so that the light source 22 is positioned in the ear canal adjacent an innermost side surface 30b of the plug 30 (i.e., the surface closest to the portion of the ear canal that is to be illuminated). The first end 20a of the body 20 is positioned externally from the ear canal adjacent an outermost side surface 30a of the plug 30 (i.e., the surface closest to the first end 20a).

In an example, the plug 30 may be made from a material that has opaque properties. Advantageously, such a material blocks any ambient light from entering the ear canal when the plug 30 and body 20 is positioned in the ear canal. Therefore, when the light source 22 is activated, all of the light from the light source 22 illuminates the area of the ear canal that is to be illuminated. Furthermore, the plug 30 blocks light from the light source 22 escaping from the ear canal to the external portion between the plug 30 and the first end 20a.

In use, it is envisaged that the plug 30 may first be introduced into the ear canal. The body 20 may then be inserted through the bore 31 such that the second end 20b of the body 20, including the light source 22, is introduced, and supported, in the portion of the ear canal that is to be illuminated. Alternatively, the body 20 may be introduced into the bore 31 of the plug 30 before insertion into the ear canal. In a further alternative example, the plug 30 may be attached to the outer surface of the body 20 before insertion into the ear canal.

The body 20 of the device 1 may also be rotated within the bore 31 of the plug 30 to allow the light source 22 to also rotate around a longitudinal axis of the body 20.

Once inserted into the ear canal, the device 1 allows for light to be focused on the portion of the ear canal that is to be illuminated. The power source may be turned on in order to activate the light source 22. In the example shown in FIG. 1, all the light from the light source 22 illuminates the portion of the ear canal that is to be operated on in all directions from the light source 22. The light source 22 effectively acts as a ‘backlight’ for the surgeon when carrying out a mastoidectomy and various other surgical procedures that involve entering the middle and inner ear canal.

FIGS. 1A and 1B show the device of FIG. 1 when it is not in the ear and when the device is not illuminating and illuminating, respectively. All of the features in relation to FIG. 1 are shown also in FIGS. 1A and 1B.

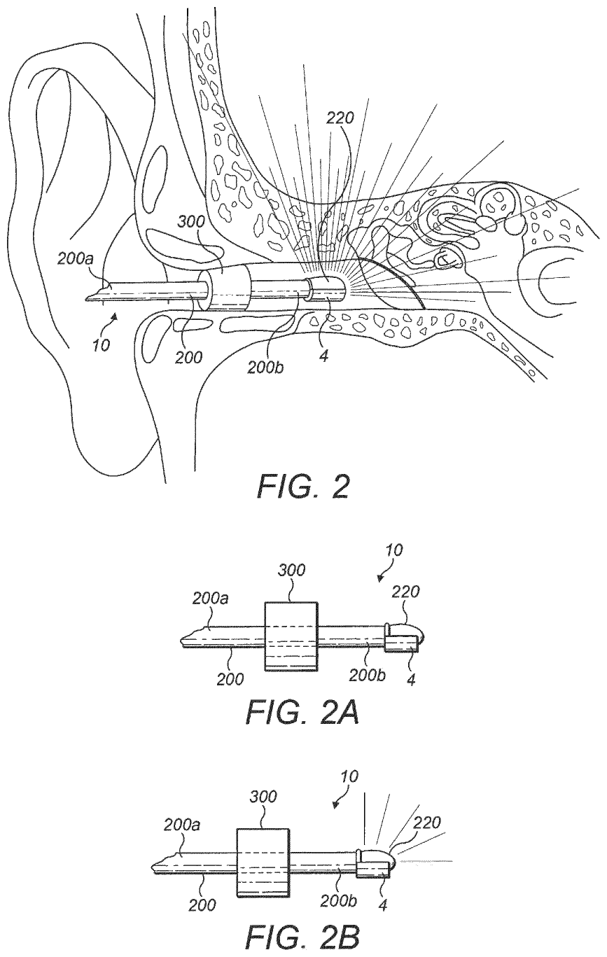

A further aspect of the invention is shown in FIG. 2. As shown in this Figure, the device 10 includes a body 200, a first end 200a, a second end 200b, a light source 220 and a plug 300. All these components are as described above in conjunction with FIG. 1.

In FIG. 2, there is also provided means for deflecting/reflecting light 4 from the light source 220. As shown in this Figure, the means for deflecting/reflecting light 4 may be an extension from the body 200 that extends from the second end 200b over a side surface of the light source 220. The means for deflecting/reflecting light 4 may be, for example as shown in FIG. 2, semi-circular in its configuration. In the example shown, the means for deflecting/reflecting light 4 has an open end to expose the end of the light source 22. The means for deflecting/reflecting light 4 may include on its inner surface reflective material to reflect the light. The means for deflecting/reflecting light 4 may then advantageously deflect and/or reflect light to a specific area in the portion of the ear canal that is to be operated on.

In the same way as body 20 described above, body 200 may be rotated within the ear canal. The surgeon may, for example, turn the first end 200a of the body 200 in order to rotate the second end 200b, which includes the means for deflecting/reflecting light 4. In other words, when the body 200 is rotated, so is the means for deflecting/reflecting light 4. Advantageously, the light emitting from the light source 220 can be deflected and/or reflected to a specific portion of the ear canal such that the surgeon can operate with better precision.

As the light may be directed in certain directions, it is important that the surgeon can determine where the light is being projected. To assist the surgeon, the body 200 may include markings on the portion of the body 200 between the plug 300 and the first end 200a. The markings may show the direction the light is projecting inside the ear canal. Therefore, when the surgeon rotates the body, the markings move in order to show the surgeon which direction the light is being deflected and/or reflected.

FIGS. 2A and 2B show the device of FIG. 2 when it is not in the ear and when the device is not illuminating and illuminating, respectively. All of the features in relation to FIG. 2 are shown also in FIGS. 2A and 2B.

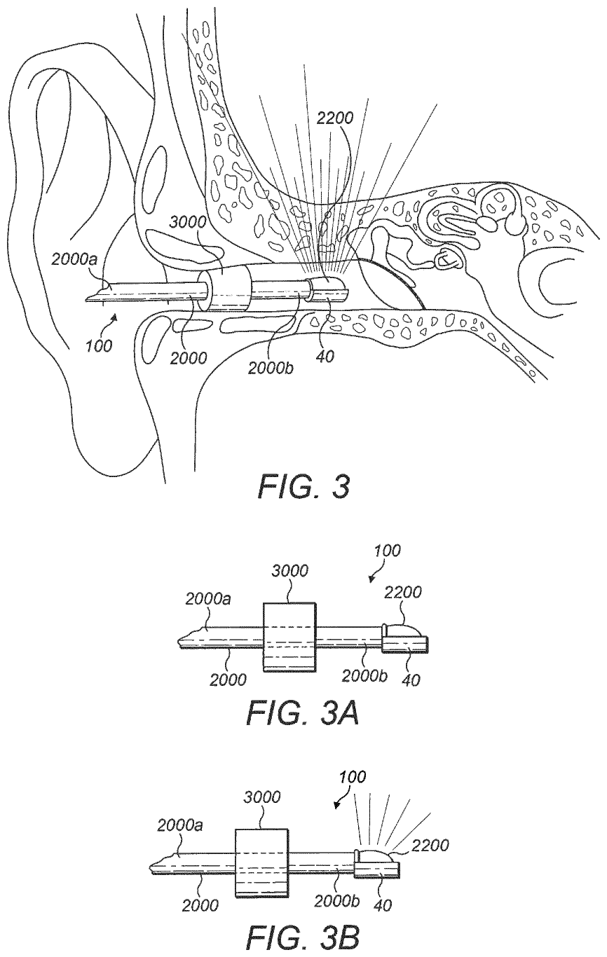

A further aspect of the invention is shown in FIG. 3. As shown in this Figure, the device 100 includes a body 2000, a first end 2000a, a second end 2000b, a light source 2200 and a plug 3000. All these components are as described above in conjunction with FIG. 1.

In FIG. 3, there is also provided means for deflecting/reflecting light 40 from the light source 2200. As shown in this Figure, the means for deflecting/reflecting light 40 may be an extension from the body 2000 that extends from the second end 2000b over a side surface of the light source 2200. The means for deflecting/reflecting light 40 may be, for example as shown in FIG. 3, semi-circular in its configuration. In the example shown, the means for deflecting/reflecting light 40 has a closed end to conceal a portion of the end (or top surface) of the light source 2200. In an example, the closed end may conceal between 1-99% of the top surface of the light source 2200. The means for deflecting/reflecting light 40 may include on its inner surface reflective material to reflect the light. The means for deflecting/reflecting light 40 may then advantageously deflect and/or reflect light to a specific area in the portion of the ear canal that is to be operated on.

In the same way as body 20 described above, body 2000 may be rotated within the ear canal. The surgeon may, for example, turn the first end 2000a of the body 2000 in order to rotate the second end 2000b, which includes the means for deflecting/reflecting light 40. In other words, when the body 2000 is rotated, so is the means for deflecting/reflecting light 40. Advantageously, the light emitting from the light source 2200 can be deflected and/or reflected to a specific portion of the ear canal such that the surgeon can operate with better precision.

FIGS. 3A and 3B show the device of FIG. 3 when it is not in the ear and when the device is not illuminating and illuminating, respectively. All of the features in relation to FIG. 3 are shown also in FIGS. 3A and 3B.

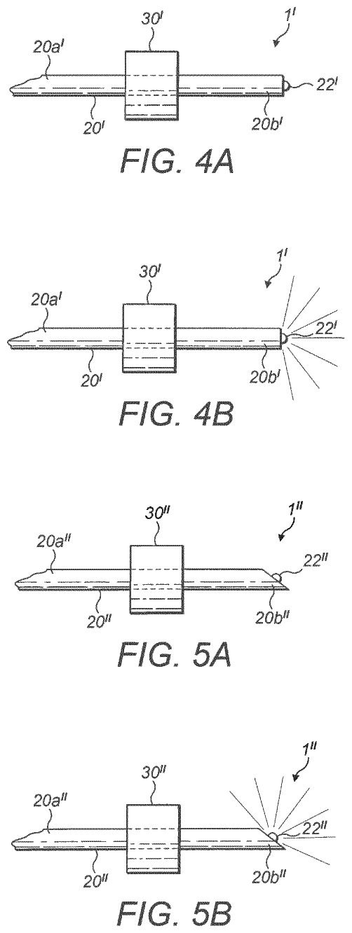

FIGS. 4A and 4B show an alternative embodiment where the light source 22′ is provided as a fibre optic. The illumination device 1′ of these Figures include a body 20′ having a first end 20a′ and a second end 20b′. The light source 22′ is located at the second end 20b′ of the body 20′. There is also provided a plug 30′ that acts in the same way as the plugs 30, 300 and 3000 discussed above. In this embodiment of the invention, the body 20′ surrounds a fibre optic cable that includes the light source 22′ at the end of the cable protruding from the second end 20b′. The light source 22′ is shown illuminated in FIG. 4B.

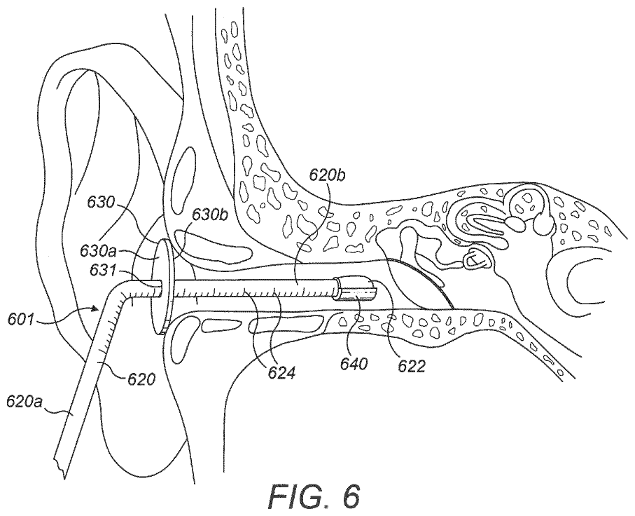

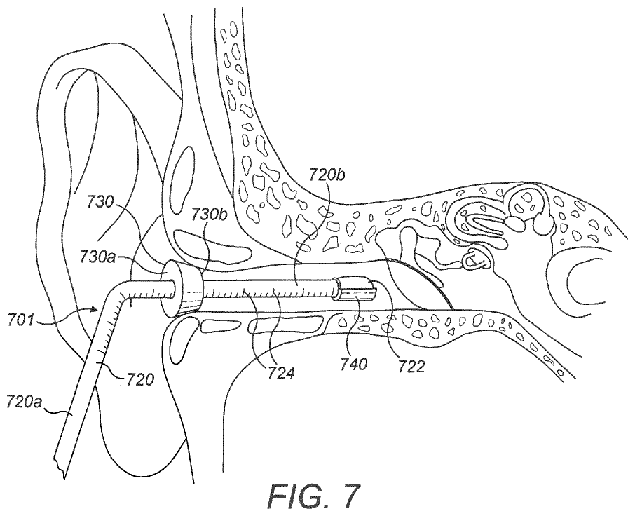

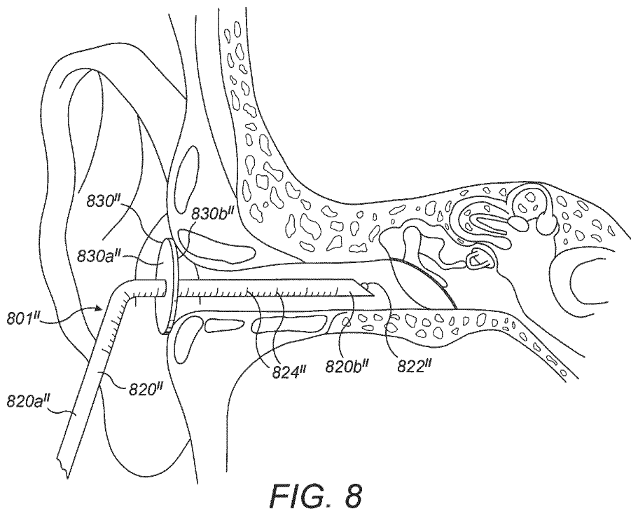

FIGS. 5A and 5B also show an alternative embodiment where the light source 22″ is provided as a fibre optic. The illumination device 1″ of these Figures include a body 20″ having a first end 20a″ and a second end 20b″. The light source 22″ is located at the second end 20b″ of the body 20″. There is also provided a plug 30″ that acts in the same way as the plugs 30, 30′, 300 and 3000 discussed above. In this embodiment of the invention, the body 20″ surrounds a fibre optic cable that includes the light source 22″ at the end of the cable protruding from the second end 20b″. As shown in FIG. 5B, the second end 20b″ of the body 20″ is provided at an angle to the longitudinal axis of the body 20″. The light source 22″ protrudes from the angled surface of the second end 20b″ of the body 20″. The angled surface provides a truncated end at the second end 20b″ of the body 20″. The truncated end may be angled for example between 10 and 80 degrees from the longitudinal axis. The light source 22″ is shown illuminated in FIG. 5B.

It is to be understood that the fibre optic described in conjunction with FIGS. 4A, 4B, 5A and 5B could be used also in conjunction with FIGS. 1-3B instead of the LEDs described above, for example.

For each of the devices discussed above, and where the body 20, 200, 2000, 20′, 20″ may freely move in and out of the ear canal, through the plug, there are provided markings on the outer surface of the body 20, 200, 2000, 20′, 20″ that show a measurement of distance (i.e., a linear scale) so that the surgeon can determine how far into the ear canal the body 20, 200, 2000 and light source 22, 220, 2200, 20′, 20″ have been inserted.

FIG. 6 shows an alternative embodiment where the plug 630 is provided external to the ear canal. The illumination device 601 of this figure includes a body 620 having a first end 620a and a second end 620b, a plug 630 and means for deflecting/reflecting light 640.

Light source 622 may be substantially similar to light sources 22, 220, 2200, 22′ and 22″ as described above. Body 620 may be substantially similar to body 20, 200, 2000, 20′ or 20″ as described above. Means for deflecting/reflecting light 640 may be substantially similar to means for deflecting/reflecting light 4 or 40 as described above.

The plug 630 comprises an outermost side surface 630a and an innermost side surface 630b. The first end 620a of the body 620 is positioned externally from the ear canal adjacent the outermost side surface 630a of the plug 630 (i.e., the surface closest to the first end 620a). Similarly, the second end 620b of the body 620 is positioned internally to the ear canal, adjacent to an innermost side surface 630b of the plug 630 (i.e., the surface closest to the second end 620b).

The plug 630 may be made from rubber or other polymeric material, foam, memory foam or silicone, for example. The plug 630 may be hard, so as to not deform upon contact with the outer ear. The plug 630 may also include a bore 631 located at its centre in order to receive the body 620. The bore 631 is sized and shaped to snugly receive the body 620. As shown in FIG. 6, the body 620 is provided through the bore 631 of the plug 630 so that the light source 622 is positioned in the ear canal adjacent an innermost side surface 630b of the plug 630 (i.e., the surface closest to the portion of the ear canal that is to be illuminated).

In an example, the plug 630 may be made from a material that has opaque properties. Advantageously, such a material blocks any ambient light from entering the ear canal when the plug 630 and body 620 is positioned in the ear canal.

Therefore, when the light source 622 is activated, all of the light from the light source 622 illuminates the area of the ear canal that is to be illuminated.

The body 620 may also comprise markings 642 on the outer surface of the body 620 to show a measurement of distance so that the surgeon can determine how far into the ear canal the body 620 and the light source 622 have been inserted.

In use, the body 620 may be inserted through the bore 631 of the plug 630. Plug 630 may then be movable along the body such that the amount of the second end 620b of the body that extends beyond the innermost side surface 630b may be varied, and therefore the distance between the light source 622 and the innermost side surface 630b may be varied as desired. When the plug 630 is at a desired distance from light source 622, it may be fixed in place. The body may then be introduced into the patient's ear canal, and inserted until the innermost side surface 630b of the plug 630 comes into contact with the outer ear. At this point, light source 622 will be at a known depth within the patient's ear canal. Advantageously, such an arrangement can prevent illumination device 601 from being inserted too far into a patient's ear canal.

The body 620 of the device 601 may also be rotated within the bore 631 of the plug 630 to allow the light source 622 to also rotate around a longitudinal axis of the body 620. The surgeon may, for example, turn the first end of the body 620 in order to rotate the second end, which includes the means for deflecting/reflecting light 640. In other words, when the body 620 is rotated, so is the means for deflecting/reflecting light 640. Advantageously, the light emitting from the light source 622 can be deflected and/or reflected to a specific portion of the ear canal such that the surgeon can operate with better precision.

When the body is orientated in such a way as desired by the surgeon, for example positioned at a desired depth within the ear with the innermost side surface 630b of the plug 630 contacting the patient's outer ear, the surgeon may secure the illumination device 601 in place. The illumination device 601 may be secured by tape fixed to the outermost side surface 630a of the plug to the ear. Alternatively, first end 620a of the body may be wrapped around the patient's ear to secure the illumination device 601 in place without need for additional fixation mechanisms. Other methods of fixation are also considered.

FIG. 7 shows yet another embodiment of the present invention. Illumination device 701 may be substantially similar to that of illumination device 601 described in conjunction with FIG. 6 above, with like reference numerals denoting corresponding features, except the plug 730 is provided within the outer ear canal. In this regard, the outermost side surface 730a of the plug 730 may be provided external to or flush to the outer ear, with the innermost side surface 730b of the plug extending into the ear canal. In this regard, the plug may hold the illumination device 701 in place without need for any additional fixation methods. Alternatively, the illumination device 701 may be further secured in place using tape, or any other suitable fixation method. The plug 730 may be shaped so as to sit at a desired depth within the ear canal. For example, the plug 730 may be tapered so that it is larger at the outermost side surface 730a than it is at the innermost side surface 730b.

Another embodiment of the present invention is shown in FIG. 8. Again, the illumination device 801″ may be substantially similar to the illumination device 601 described above in conjunction with FIG. 6 with like reference numerals denoting corresponding features, except light source 822″ is a fibre optic cable similar to that depicted in FIGS. 5A and 5B.

Although the invention has been described in terms of preferred embodiments as set forth above, it should be understood that these embodiments are illustrative only and that the claims are not limited to those embodiments. Those skilled in the art will be able to make modifications and alternatives in view of the disclosure which are contemplated as falling within the scope of the appended claims.

1.-13. (canceled)

14. An illumination device for ear surgery, said illumination device comprising:
a body having a first end and a second end;
a light source provided at the second end of the body; and
means for supporting the body and the light source in a patient's ear canal, wherein means for deflecting/reflecting light is provided at the second end of the body and along a side surface of the light source.

15. The illumination device of claim 14, wherein the means for supporting said body is provided around a portion of the body between the first end and the second end.

16. The illumination device of claim 14, wherein the body is provided in a bore of the means for supporting the body and the light source.

17. The illumination device of claim 14, wherein the body has a longitudinal axis and wherein the body is configured to rotate around the longitudinal axis.

18. The illumination device of claim 14, wherein the means for deflecting/reflecting light covers a portion of a top surface of the light source.

19. The illumination device of claim 18, wherein the means for deflecting/reflecting light covers between 1-99% of the top surface of the light source.

20. The illumination device of claim 14, wherein the first end of the body is attached to a power source to activate the light source.

21. The illumination device of claim 14, wherein the means for supporting said body and said light source is a plug.

22. The illumination device of claim 21, wherein the plug is made of a polymeric material.

23. The illumination device of claim 14, wherein the light source is an LED or a fiber optic.

24. The illumination device of claim 14, wherein the means for supporting said body is movable with respect to the body.

25. The illumination device of claim 14, wherein the means for supporting the body is configured to be positioned external to the patient's ear canal.

26. A kit comprising:
a plug adapted to fit within or external to a patient's ear canal, the plug having a bore through its center; and
a body having a first end and a second end, the body having a light source at its second end; and
wherein a means for deflecting/reflecting light is provided at the second end of the body and along a side surface of the light source; and
wherein the body is adapted to be inserted through the bore of the plug.
